(19) United States
(12) Patent Application Publication
(10) Pub. No.: US 2021/0219964 A1
(43) Pub. Date: Jul. 22, 2021

(54) POWERED BONE-GRAFT HARVESTER

(71) Applicants: Stephen F. Conti, (US); MicroAire Surgical Instruments, LLC, Charlottesville, VA (US)

(72) Inventors: Stephen F. Conti, Pittsburgh, PA (US); Dwayne Batten, Charlottesville, VA (US)

(21) Appl. No.: 17/151,422

(22) Filed: Jan. 18, 2021

Related U.S. Application Data
(60) Provisional application No. 62/962,366, filed on Jan. 17, 2020.

(51) Int. Cl. A61B 10/02

(52) U.S. Cl. CPC A61B 10/025; A61B 2010/0208

(57) ABSTRACT

A bone graft harvester and method of use are described which produces bone grafts of superior quality to existing techniques. The bone graft harvester is driven in a reciprocating and impacting fashion when placed against bone to be fashioned at a rate of 500 to 20000 impacts per minute and with non-rotating longitudinal movements of 0.5 to 5 mm. Bone is collected in a hollow core of the hollow core coring tool of the bone graft harvester and can be retrieved therefrom for bone grafting procedures. Preferably the hollow core is tapered to allow expansion of the bone in the coring tool so as to better hold bone which is retrieved from the harvesting site.

### FIELD OF THE INVENTION

Embodiments herein are generally related to medical procedures involving tissue harvesting and, in particular, bone harvesting with coring tools.

### BACKGROUND

During certain medical procedures, it may be necessary to harvest autologous bone graft using a manual bone-coring device.

The manual coring method of bone-graft harvesting can take 15-20 minutes during a typical bone-fusion procedure, such as an ankle arthrodesis. One common method is performed by holding a cylindrical coring device with a T-shaped handle, and pressing and twisting the device into and through the donor-site cortical bone, to reach the softer cancellous bone within, for the purpose of filling the collection cylinder with cancellous bone tissue.

In some cases the coring device would include small teeth or serrations around one end of the cylinder, and a surgeon would twist the device to, in effect, supply a rotary sawing motion for cutting into the bone tissue. It is usually necessary to repeat the pushing and twisting movement three or four times to obtain a sufficient amount of graft material, and it usually provides a loose slurry of bone particulate.

Prior art tools and techniques for harvesting bone grafts often yield “mushy” cores. For a number of medical procedures and applications a bone graft with higher quality is desirable for better clinical outcomes.

### SUMMARY

In an embodiment of the invention, surgical device for harvesting bone has a hollow core coring tool that is driven in a reciprocating and impacting manner by a motorized driver often referred to as a powered handpiece. The hollow core coring tool has a bone engaging end and second end connected or connectable to the motorized driver. Connection can be direct or through a shank assembly. The powered handpiece is configured to, in a reciprocating and impacting manner, move the hollow core coring tool longitudinally 0.25 to 5.0 mm from a first position to a second position wherein the first position relatively farther from the powered handpiece at a rate of 500 to 20000 impacts per minute.

In another embodiment of the invention, a surgical method for harvesting bone involves positioning a hollow core coring tool with a bone-engaging end on a bone tissue, and pressing the bone-engaging end of the hollow core coring tool against the bone tissue while repetitively impacting the bone tissue with the bone-engaging end of the hollow core coring tool. The impacting is performed at 500 to 20000 impacts per minute, and is performed by longitudinally moving the bone-engaging end of the hollow core coring tool 0.25 to 5.0 mm from a first position to a second position in a reciprocating motion wherein said first position is relatively closer to the bone tissue than the second position. The bone core is collected within the hollow core of the hollow core coring tool as the hollow core coring tool is pressed into the bone tissue during the pressing step. After the bone core is collected, it is retrieved from the hollow core of the hollow core coring tool and is usable for bone grafting and for other applications. Good results are achieved in autologous retrieval and reuse of the bone core.

According to some embodiments, the powered-instrument method of harvesting bone grafts can take 1-3 minutes during an ankle arthrodesis procedure. This is safer for the patient by reducing time under anesthesia. In addition, it provides cost savings compared to the manual method. In the example of an ankle arthrodesis, the harvest is performed by holding the motorized driver (e.g., powered handpiece) in one or two hands and plunging the coring attachment (e.g., the hollow core coring tool) into the calcaneus bone to extract a graft. The resulting graft is more uniform and more dense than manually obtained grafts, and provides an ample supply of graft material in one pass, which material aids in formation of a scaffold in the voids of the fusion.

According to an aspect of some embodiments, a device comprises a motorized handpiece configured for accepting a variety of cutting or coring attachments to be used for applications previously requiring manual instruments. Such device yields bone grafts of superb bone densities acceptable for use in a variety of medical procedures.

According to an aspect of some embodiments, the motorized handpiece is configured to receive as an attachment a cylindrical coring device for harvesting bone graft. Coring devices may vary in size, and may have a length of 5 mm-100 mm and a width large enough to provide a hollow core interior of a diameter or cross-sectional opening of 2 mm to 20 mm. Cylindrical bone cores may be obtained when the hollow core is circular in shape; however, other configurations, e.g., polygonal (e.g., six sided or eight sided) or elliptical openings may also be used. Preferably at least a portion of the hollow core coring tool is tapered. For example, it may be tapered at the bone engaging end and be relatively cylindrical thereafter, however, to better engage and hold the bone core, it is preferred that the taper extend more than fifty percent of the length of the hollow core, and in some applications, the entire length of the hollow core. With the taper, as the diameter decreases, bone which is collected therein will be held more tightly.

The handpiece drives the attached coring device akin to a miniature jackhammer at a rate of 500 to 20,000 impacts per minute, e.g., 4,000-12,000 strikes per minute. Preferably the speed of impact can be varied and be selectable using a speed setting. Impact-reciprocation is achieved through a hammer action within the motorized driver that activates only when the cutting accessory is pushed onto the bone (causing the cutting accessory to retract toward the instrument and then springing it forward again), thereby reducing the potential for the cutting accessory to skip and jump on the bone as it might otherwise if the reciprocating action was constant.

In a preferred embodiment, the hammering effect of the motorized handpiece will only engage the distal end of the device connected to the cutting accessory if the user is actively pushing the device toward the target bone. The distal end of the device does not get retracted when the hammer mechanism itself gets retracted. The distal end of the unit is held distally away from the hammer mechanism with a spring or other resilient member and will only be retracted back by forward force of the user on the device drawing the distal end of the handpiece into the hammer mechanism. The harder the user pushes into the target bone will result in a larger percentage of the hammer force being translated into forward motion into the bone (the bigger the hammer effect). One example of a suitable mechanism is described in U.S. Pat. No. 4,298,074, which is herein incorporated by reference.

According to an aspect of some embodiments, exemplary devices are well suited for bone grafting during fusions. The device rapidly and precisely harvests bone grafts through the impact-reciprocation action of the cutting accessory, which action has a very short stroke, and which might be viewed as “vibration”.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1A is a perspective view of a cylindrical coring device.

FIG. 1B is a section view of an exemplary coring device.

FIG. 1C is a section view of another exemplary coring device.

FIG. 1D is an enlarged view of the coring end of the coring device.

FIG. 2 is a schematic setting forth a surgical method of harvesting a bone sample.

FIG. 3 is a pictorial image of an exemplary handpiece configured for attachment with the coring device.

FIG. 4 is a photograph of two exemplary coring devices of alternate sizes and bone grafts harvested with each.

FIG. 5 is a bone-harvesting device with quick attachment.

FIG. 6 is a quick attach shank.

FIG. 7 is a quick attach shank assembly.

FIG. 8 is a quick attach shank with coring device.

### DETAILED DESCRIPTION

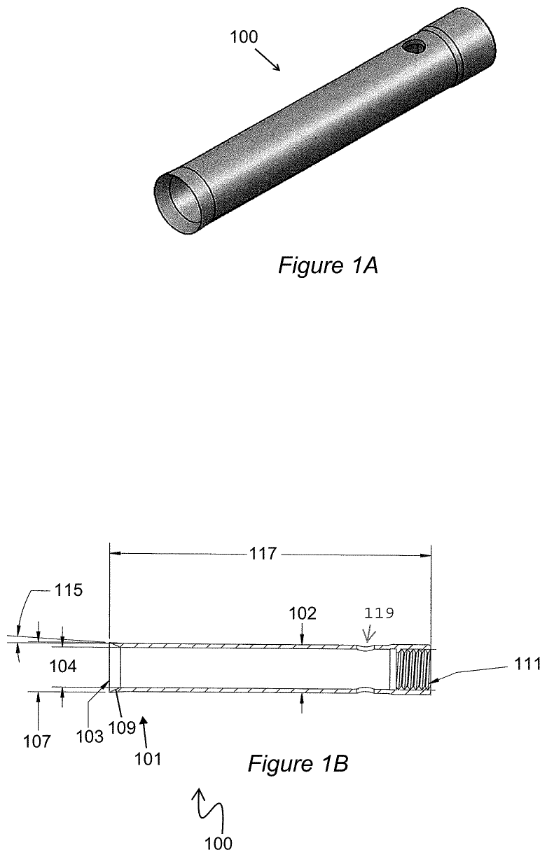

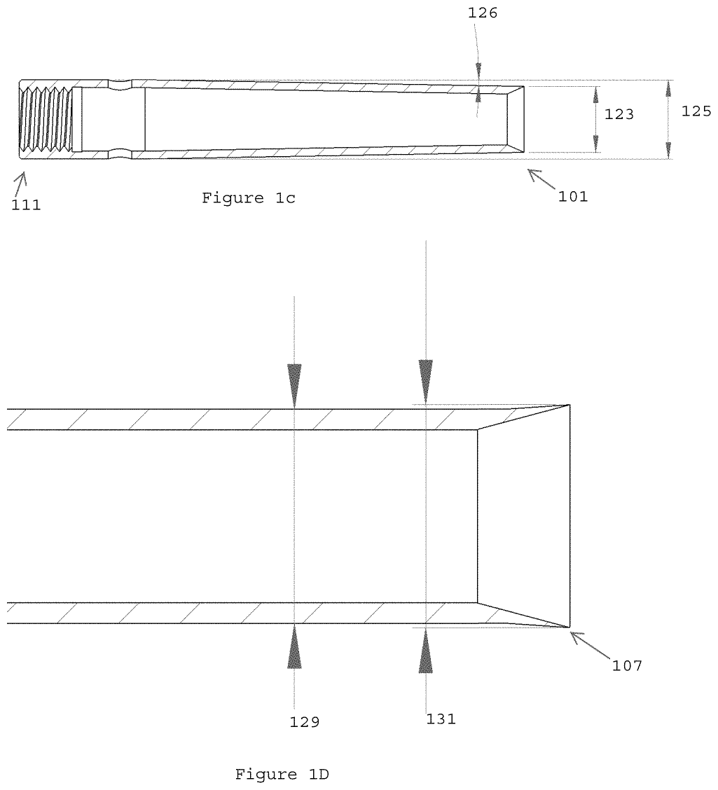

FIGS. 1A and 1B show an exemplary bone harvesting device which includes a coring device, referred to as a hollow core coring tool 100, that is attached or attachable to an impact-reciprocating handpiece. Unlike a manual cylindrical coring device, the powered device penetrates the bone in a manner that allows for great compaction and higher density yields. In some embodiments, the shape may be fully tubular. The shape of the cutting edge is preferably a complete circle as opposed to an arc, for example. The three-dimensional shape of the distal cutting end may have a variety of forms including but not limited to a cylinder, tube, or pipe.

The hollow core coring tool 100 can vary in size and shape depending on the application. The length may range from 5 mm to 100 mm. The width/diameter may range from 2 mm to 20 mm. Preferably the core diameter or cross-section may range from 4 mm to 10 mm. The opening at the bone engaging end 103 may be circular, elliptical or polygonal.

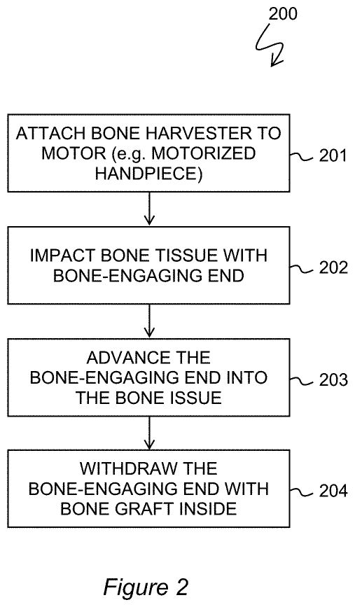

FIG. 2 shows a surgical method 200 for harvesting bone. At block 201 the accessory or attachment for bone harvesting (i.e., the hollow core coring tool 100) is attached to a motor, such as motorized handpiece, capable of supplying an impact-reciprocating motion to the accessory or attachment. At block 202 the bone tissue is impacted with the bone-engaging end of the harvester. In operation, reciprocal movement of the bone harvesting tool with impaction only proceeds when the tool 100 is in contact with the bone. In this way movement and destruction of nearby tissues is avoided. Reciprocal impacting movements preferably proceeds at 500 to 20,000 impacts per minute, and the hollow core coring tool is 100 is moved toward the bone a longitudinal distance of 0.25 mm to 5.0 mm per cycle. In a preferred embodiment, the displacement of the bone engaging end of the hollow core coring tool relative to the powered handpiece is 2.0 mm or less. Preferably, the impact rate or speed is selectable by the surgeon between 4,000 and 12,000 impacts per minute. Slower speeds may allow more mass to be incorporated into the hammer mechanism, since it is harder to quickly move significant mass. Thus, one may incorporate different amounts of mass and couple that with specific speed ranges. More mass may allow for more hammer force which may be good for harder bone. In addition, faster action and less stroke would generally be better for more precision cutting. By contrast, slower and longer action may be better for larger bone removal. In some embodiments, the displacement distance may be selectable by the surgeon between 0.5 mm and 2.0 mm.

A user such as surgeon preferably simultaneously advances the tool manually into the bone tissue at block 203. Generally, the user will advance the instrument such that the distal end reaches beyond the cortical bone exterior to the cancellous tissue within the bone. The instrument may be advanced through the bone to the opposite side, but this is not necessary unless desired by the surgeon. Finally, the instrument is withdrawn at block 204 with the bone graft inside. To separate a graft from the rest of the bone, a twisting motion can be employed, but it is not actually required. The bottom of the core naturally separates from the rest of the bone. In preferred embodiments, the powered handpiece does not rotate the hollow core coring tool 100 during reciprocal motion (i.e., there is only one degree of motion for the hollow core coring tool imparted by the handpiece).

Returning to FIG. 1B there is shown a cross-section of the hollow core coring tool 100 taken along a bisecting longitudinal plane. The hollow core coring tool 100 has an inner diameter (ID) 104, which preferably ranges from 2 to 20 mm and is selected based on the application, and an outer diameter (OD) 102. In FIG. 1B, a countersink 109 at a distal part 101 causes the ID 104 to gradually enlarge approaching the distal end until equaling the OD at a distal cutting edge 103. That is, the countersink provides a taper inward into the hollow bone core receiving volume. At least the distal part 101, and up to an entirety of the device 100, has a hollow center sized to accommodate a bone graft. As discussed above, although the illustrated device has a circular cross-section, the cross-sectional profile of the bone-engaging end of embodiments may vary, e.g., shapes may include a hollow polygon, ellipse, or circle. The distal end may have a flare 107 such that the OD gradually increases. The flare may have an angle 115. The distal cutting edge 103 may be ground to sharp. The flare 107 is advantageous as it causes the hole or tunnel made in the bone tissue to be of slightly greater diameter than OD 102. The clearance of OD 102 reduces contact between the outer walls of the coring device 100 and bone tissue alongside the outer walls. Since the coring device 100 is made to reciprocate at very high rates (e.g., several thousand strikes per minute), reducing contact between the coring device 100 and adjacent bone tissue outside of the coring device advantageously reduces friction-induced heating of the bone tissue. Also shown in FIG. 1B are vent holes 119 which prevent air from being trapped inside the coring tool. Allowing the outward flow of air may prevent the build up of pressure behind the bone core as bone fills the coring from the distal end.

FIG. 1C is substantially the same as FIG. 1B except the hollow core is tapered outwardly from the distal, bone engaging, end (the first end) 101 to the proximal, powered handpiece engaging, end (the second end) 111. As can be seen the opening 123 at the distal end 101 is smaller than the diameter 125 of the coring tool towards its proximal end as is indicated by arrow 126. The taper may allow the bone material to naturally expand once captured in the coring tool and be trapped by the taper in the coring device.

FIG. 1D shows an exemplary embodiment of the flare 107 which may be used at the distal, bone engaging, end 101 of the coring tool. As noted by the arrows 129 and 131, the diameter 129 of the coring tool at one or more points towards the proximal end is smaller, than the diameter 131 at the flare 107. In addition to the advantages discussed above, the flare 107 will make it easier to remove the coring tool from the bone after advancing into bone.

The coring device 100 may have a total length 117 of 4 inches or less, 3 inches or less, or 2 inches or less, or some other length. The length of a particular coring device 100 to be used for a particular patient or procedure may be selected based on the length of bone graft required and the source bone from which it is to be taken. The OD and ID of a specific coring device 100 to be used may also be selected based on the size of the bone graft required. Bone grafts harvested with a coring device 100 may have a diameter as small as 2 mm or as large as 10 mm or more, for example. The ID 104 of a coring device 100 may similarly be as small as 2 mm or as large as 20 mm, plus appropriate tolerances. Some embodiments may comprise kits of differently sized coring devices 100.

The coring device 100 further includes an attachment means 111 at a proximal end for attaching to a motorized handpiece. The attachment means 111 may be a threaded shaft as shown in FIG. 1B.

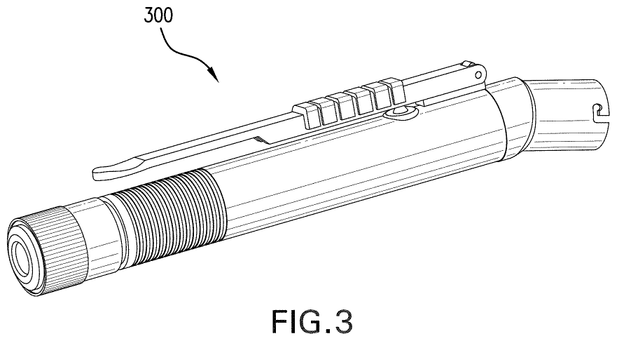

FIG. 3 shows a motorized handpiece 300 based on U.S. Pat. No. 4,298,074 to Mattchen the complete contents of which is herein incorporated by reference. The motorized handpiece 300 is an example of a handpiece which can provide the reciprocal longitudinal movement of the hollow core coring device 100 at the impaction rate contemplated herein. In preferred embodiments, the motorized handpiece may be driven by electrical power as opposed to air pressure discussed in Mattchen. The hollow core coring device 100 is removably attached or attachable to the powered handpiece 300. The powered handpiece 300 may have complementary attachment means (not shown) adapted to connect with attachment means 111 of the coring device 100.

The handpiece 300 is motorized and configured to reciprocate the attached coring device. The coring device is driven with an impact motion, in contrast to either a drilling motion or rotary motion. That is, the handpiece 300 may in some cases be configured or configurable to not supply rotation about a longitudinal axis of the coring device, i.e., the axis along which the handpiece oscillates the coring device. The powered handpiece may be configured to drive the bone harvesting attachment or accessory at a rate of at least 500 to 20,000 impacts per minute, e.g., 4000 impacts per minute to 12,000 impacts per minute, for example. The powered handpiece may cause a reciprocation that results in each impact cycle resulting in a displacement (e.g., movement in the longitudinal direction of the hollow core coring tool 100 from a first position to a second position) of the hollow core coring tool 100 of 0.5 mm to 2.0 mm relative to the handpiece along its longitudinal axis (e.g., movement in the longitudinal direction of the hollow core coring tool 100 from a first position to a second position). The handpiece may accept not just the coring device 100 but a variety of different attachments, including but not limited to osteotome, chisel, gouge, and bone-graft harvester.

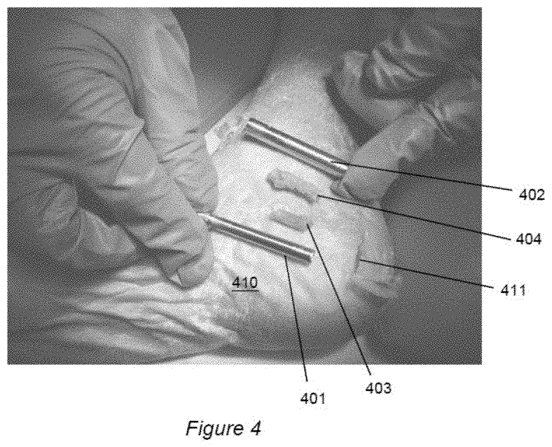

FIG. 4 is a photograph of various hollow core coring tools referred to as coring devices 401 and 402. Coring device 401 has a smaller ID than coring device 402. Bone graft 403 was harvested using coring device 401, whereas bone graft 404 was harvested using coring device 402. The bone grafts were obtained through an incision 411 of a foot 410 of a patient. The photograph shows the excellent densities of bone grafts 403 and 404.

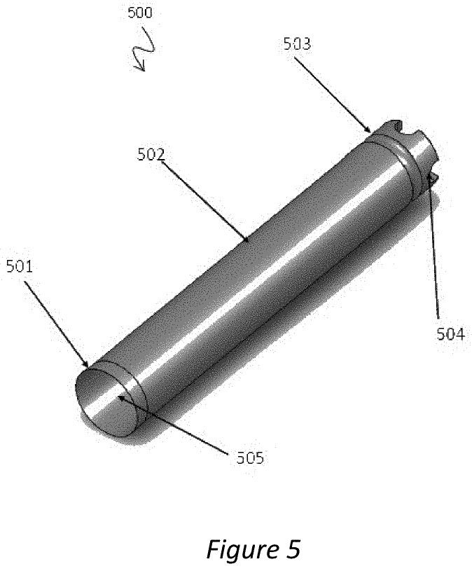

FIG. 5 is a coring tool 500 configured for quick attachment. The coring tool 500 includes a cutting edge 401, a cylindrical shaft 502, a ball groove 503, and anti-rotation slots 504. The ball groove 503 and anti-rotation slots 504 are configured to allow the tool 500 to be joined with a quick attach shank. The tool 500 further includes a sample funnel 505, which is also a storage reservoir.

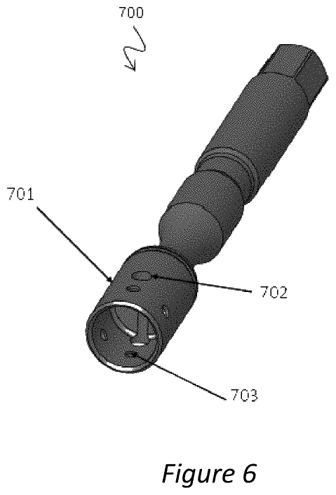

FIG. 6 is a quick attach shank 700. The quick attach shank 700 comprises an end part 701 into which an end effector such as a coring device 500 (FIG. 5) may be inserted. The quick attach shank 700 includes an anti-rotation pin hole 702 and steel ball holes 703. The quick release system allows surgeons to quickly remove and attach varying cutters to save them time and aggravation, by sparing the need for wrenches and tooling to change out attachments (as is most common with prevailing tools).

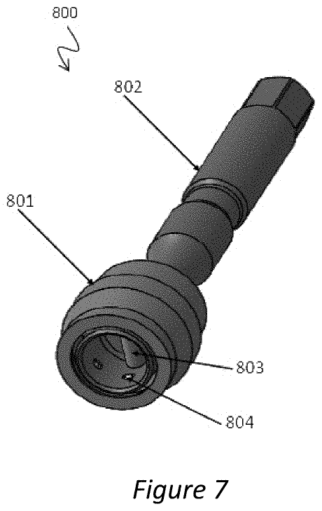

FIG. 7 is a quick attach shank assembly 800. The quick attach shank assembly 800 includes a quick attach shank 803 (corresponding to quick attach shank 700 of FIG. 6), a quick attach collar 801, an anti-rotation pin 803, and steel balls 804.

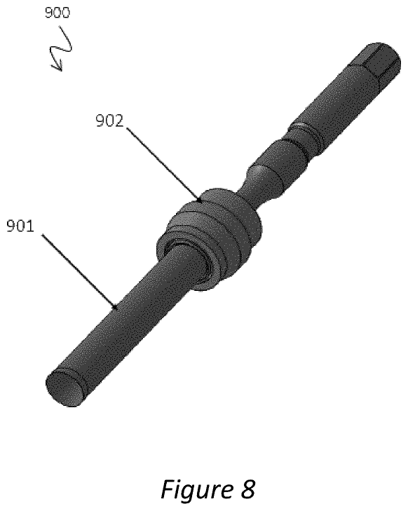

FIG. 8 is an assembly 900 comprising a coring device 901 (corresponding to 500 in FIG. 5) and a quick attach shank assembly 902 (corresponding to 800 in FIG. 7).

Exemplary devices are well suited for bone grafting during fusions and rheumatoid surgery. Bone grafting is a surgical procedure that replaces missing bone in order to repair bone fractures that are extremely complex, pose a significant health risk to the patient, or fail to heal properly. Bone grafts may be harvested from a variety of sites included but not limited to the ankle and foot.

While exemplary embodiments of the present invention have been disclosed herein, one skilled in the art will recognize that various changes and modifications may be made without departing from the scope of the invention as defined by the following claims.

What is claimed is:

1. A surgical method for harvesting bone, comprising:
positioning a hollow core coring tool with a bone-engaging end on a bone tissue;
pressing the bone-engaging end of the hollow core coring tool against the bone tissue and repetitively impacting the bone tissue with the bone-engaging end of the hollow core coring tool, wherein impacting is performed at 500 to 20000 impacts per minute, and wherein impacting is performed by longitudinally moving the bone-engaging end of the hollow core coring tool 0.5 to 2.0 mm from a first position to a second position in a reciprocating motion wherein said first position is relatively closer to the bone tissue than the second position;
collecting a bone core within the hollow core of the hollow core coring tool as the hollow core coring tool is pressed into the bone tissue during the pressing step; and
retrieving the bone core from the hollow core of the hollow core coring tool.

2. The surgical method of claim 1 wherein the impacting is performed without rotation of the hollow core coring tool during the reciprocating motion.

3. The surgical method of claim 1 wherein the hollow core of the hollow core coring tool has a diameter or cross-sectional opening of 2 to 20 mm.

4. The surgical method of claim 1 wherein at least a portion of the hollow core of the hollow core coring tool is tapered from the bone engaging end of the hollow core coring tool to a second end of the hollow core coring tool.

5. The surgical method of claim 1 wherein the hollow core of the hollow core coring tool has a shape selected from the group consisting of circular, polygonal, and elliptical.

6. The surgical method of claim 1 wherein the distal end of the coring tool includes a flare that is larger than an outer diameter of a main body of the coring tool.

7. The surgical method of claim 1 wherein the bone coring tool includes one or more vent holes at a proximal end of the coring tool.

8. A surgical device for harvesting bone, comprising:
a hollow core coring tool having a bone-engaging end and a second end, wherein the hollow core of the coring tool is sized to accommodate a bone core therein; and
a powered handpiece connected or connectable to the hollow core coring tool, wherein the powered and piece is configured to, in a reciprocating and impacting manner, move the hollow core coring tool longitudinally 0.5 to 2.0 mm from a first position to a second position wherein the first position relatively farther from the powered handpiece at a rate of 500 to 20000 impacts per minute.

9. The surgical device of claim 8 wherein the rate of impact is selectable and adjustable from 500 to 20000 impacts per minute.

10. The surgical device of claim 8 wherein the rate of impacts is between 4000 and 12000 impacts per minute.

11. The surgical device of claim 8 wherein a length of longitudinal movement of the hollow core coring tool is selectable and adjustable from 0.5 to 2.0 mm.

12. The surgical device of claim 8 wherein a length of longitudinal movement of the hollow core coring tool is 1.0 to 2.0 mm.

13. The surgical device of claim 8 wherein the powered handpiece does not rotate the hollow core coring tool during reciprocating movement.

14. The surgical device of claim 8 wherein the powered handpiece is connected to the second end of the hollow core coring tool.

15. The surgical device of claim 8 further comprising a shank assembly, wherein the powered handpiece is connected to the second end of the hollow core coring tool by the shank assembly.

16. The surgical device of claim 8 wherein the hollow core of the hollow core coring tool has a diameter or cross-sectional opening of 2 to 20 mm.

17. The surgical device of claim 8 wherein at least a portion of the hollow core of the hollow core coring tool is tapered from the bone engaging end of the hollow core coring tool to the second end of the hollow core coring tool.

18. The surgical device of claim 8 wherein the hollow core coring tool is tapered for more than fifty percent of a longitudinal length of the hollow core coring tool.

19. The surgical device of claim 8 wherein the hollow core coring tool is tapered the bone engaging end.

20. The surgical device of claim 8 wherein the hollow core of the hollow core coring tool has a shape selected from the group consisting of circular, polygonal, and elliptical.

21. The surgical method of claim 8 wherein a distal end of the coring tool includes a flare that is larger than an outer diameter of a main body of the coring tool.

22. The surgical method of claim 8 wherein the bone coring tool includes one or more vent holes at a proximal end of the tool.
